(19) United States
(12) Patent Application Publication
(10) Pub. No.: US 2025/0296974 A1
(43) Pub. Date: Sep. 25, 2025

(54) MODIFIED FOLLICLE-STIMULATING HORMONE AND METHODS OF USING THE SAME

(71) Applicant: The Regents of the University of Colorado, a body corporate, Denver, CO (US)

(72) Inventor: T. Rajendra KUMAR, Aurora, CO (US)

(21) Appl. No.: 18/863,230

(22) PCT Filed: May 8, 2023
(86) PCT No.: PCT/US2023/066734

§ 371 Date: Nov. 5, 2024

Related U.S. Application Data
(63) Continuation-in-part of application No. 17/662,312, filed on May 6, 2022.
(60) Provisional application No. 63/364,299, filed on May 6, 2022.

(51) Int. Cl. C07K 14/59; A61K 38/00

(52) U.S. Cl. CPC C07K 14/59; A61K 38/00

(57) ABSTRACT

The present inventive concept is related to a modified follicle-stimulating hormone (FSH), in particular, a hypo-glycosylated form of FSH, as well as compositions and formulations including, nucleic acids encoding, cell lines expressing, and methods of using the hypo-glycosylated FSH as disclosed herein. These methods of use include methods of treating infertility, methods of inducing follicle growth and/or maturation, methods of inducing oocyte/egg growth and/or maturation, methods of stimulating sex steroid secretion, methods of treating or preventing bone density loss, and methods of treating or preventing fatty tissue accumulation.

### CROSS-REFERENCE TO RELATED APPLICATIONS

The present application claims the benefit of U.S. patent application Ser. No. 17/662,312, filed May 6, 2022, and U.S. Provisional Patent Application No. 63/364,299, filed May 6, 2022, the disclosures of each of which are incorporated herein by reference in their entireties.

### STATEMENT REGARDING FEDERAL SUPPORT

This invention was made with government support under grant number AG029531 awarded by National Institutes of Health. The government has certain rights in the invention.

### STATEMENT REGARDING ELECTRONIC FILING OF A SEQUENCE LISTING

A Sequence Listing XML file, submitted in accordance with 37 C.F.R. §§ 1.831-835, entitled 151077-00045WO_ST26.xml, generated May 2, 2023, 19,720 bytes in size, and filed electronically, is provided in lieu of a paper copy. The Sequence Listing is incorporated herein by reference into the specification for its disclosures.

### FIELD OF THE INVENTION

The present invention relates to compositions including a recombinant form of Follicle-Stimulating Hormone (FSH), and methods of using the same for treating and/or preventing metabolic disorders such as: infertility; bone density loss; and/or adipose tissue accumulation.

### BACKGROUND OF THE INVENTION

Follicle-Stimulating Hormone (FSH) is a pituitary-derived heterodimeric glycoprotein that is essential for female reproduction. It consists of a hormone-specific beta-subunit and an alpha-subunit that is common to other glycoprotein hormones in pituitary and placenta. Female mice or women lacking the FSH ligand as a result of a null mutation in the FSH beta-subunit are infertile, do not produce estrogen and their ovarian follicle development is arrested. For various genetic and other metabolic reasons, women do not also produce sufficient endogenous FSH and therefore require exogenous FSH supplementation for optimal ovarian follicle growth and estrogen production, a common practice in artificial reproductive technology (ART) induction protocols. However, the response rate in many women to recombinant FSH is very poor. Currently, clinical grade recombinant human FSH is expressed in Chinese Hamster Ovary (CHO) cells which have a different glycosylation machinery compared to that in pituitary.

The pituitary and serum levels of FSH change with aging and it was discovered that aging also changes the glycosylation signature specifically on the FSH beta-subunit resulting in different types of FSH glycoforms whose abundance is age-specific. It was discovered that during young and normal reproductive cycles, women produce more of a hypo-glycosylated FSH, designated FSH²¹, whereas older women produce fully-glycosylated FSH²⁴. FSH²¹ was found to be more biologically active, and binds FSH receptors on ovarian cells, and cell lines more effectively than FSH²⁴.

The commercial potential of recombinant human FSH products for use in ART protocols is enormous, exceeding 1.5 billion US dollars worldwide. As such, there is a need for improved products, such as improved FSH products, and methods of using such products in ART protocols and treatment of infertility. In addition, there is also presently a need for improved products, such as improved FSH products, and methods of using such products for treatment and prevention of bone density loss and fatty tissue accumulation.

### SUMMARY OF THE INVENTION

The present inventive concept utilizes a hypo-glycosylated form of FSH, which provides the basis of the compositions and methods of the present inventive concept.

Thus, in an aspect of the inventive concept, provided is a recombinant Follicle-Stimulating Hormone (FSH) beta-subunit, wherein a glycosylation site on the FSH beta-subunit is substituted with an amino acid at which a glycan is not and/or cannot be attached.

In another aspect of the present inventive concept, provided is a recombinant Follicle-Stimulating Hormone (FSH) including an FSH alpha-subunit and an FSH beta-subunit, wherein a glycosylation site on the FSH beta-subunit is substituted with an amino acid at which a glycan is not and/or cannot be attached.

In another aspect of the present inventive concept, provided is a nucleic acid including a sequence encoding an FSH beta-subunit, wherein a glycosylation site on the FSH beta-subunit is substituted with an amino acid at which a glycan is not and/or cannot be attached.

In another aspect of the present inventive concept, provided is an expression vector comprising the nucleic acid including a sequence encoding an FSH beta-subunit, wherein a glycosylation site on the FSH beta-subunit is substituted with an amino acid at which a glycan is not and/or cannot be attached, and a heterologous promoter operably associated therewith.

In another aspect of the present inventive concept, provided is a cell line comprising an expression vector comprising the nucleic acid including a sequence encoding an FSH beta-subunit, wherein a glycosylation site on the FSH beta-subunit is substituted with an amino acid at which a glycan is not and/or cannot be attached, and a heterologous promoter operably associated therewith, and a cell line expressing an FSH beta-subunit or recombinant FSH including an FSH beta-subunit, wherein a glycosylation site on the FSH beta-subunit is substituted with an amino acid at which a glycan is not and/or cannot be attached.

In another aspect of the present inventive concept, provided is a pharmaceutical composition or formulation including an FSH beta-subunit or recombinant FSH including an FSH beta-subunit, wherein a glycosylation site on the FSH beta-subunit is substituted with an amino acid at which a glycan is not and/or cannot be attached, and a pharmaceutically acceptable carrier.

In another aspect of the present inventive concept, provided is a method of treating infertility in a subject including administration of a composition or formulation including an FSH beta-subunit or recombinant FSH including an FSH beta-subunit, wherein a glycosylation site on the FSH beta-subunit is substituted with an amino acid at which a glycan is not and/or cannot be attached to a subject in need thereof.

In another aspect of the present inventive concept, provided is a method of inducing follicle growth and/or maturation comprising administration of a pharmaceutical composition or formulation including an FSH beta-subunit or recombinant FSH including an FSH beta-subunit, wherein a glycosylation site on the FSH beta-subunit is substituted with an amino acid at which a glycan is not and/or cannot be attached to a subject in need thereof.

In another aspect of the present inventive concept, provided is a method of inducing oocyte/egg growth and/or maturation comprising administration of a pharmaceutical composition or formulation including an FSH beta-subunit or recombinant FSH including an FSH beta-subunit, wherein a glycosylation site on the FSH beta-subunit is substituted with an amino acid at which a glycan is not and/or cannot be attached to a subject in need thereof.

In another aspect of the present inventive concept, provided is a method of stimulating secretion of a sex steroid including administering a therapeutically effective amount of a pharmaceutical composition or formulation including an FSH beta-subunit or recombinant FSH including an FSH beta-subunit, wherein a glycosylation site on the FSH beta-subunit is substituted with an amino acid at which a glycan is not and/or cannot be attached to a subject in need thereof.

In another aspect of the present inventive concept, provided is a method of treating and/or preventing loss of bone density including administering a therapeutically effective amount of a composition or formulation including an FSH beta-subunit or recombinant FSH including an FSH beta-subunit, wherein a glycosylation site on the FSH beta-subunit is substituted with an amino acid at which a glycan is not and/or cannot be attached to a subject in need thereof.

In another aspect of the present inventive concept, provided is a method of treating and/or preventing fatty tissue accumulation including administering a therapeutically effective amount of a composition or formulation including an FSH beta-subunit or recombinant FSH including an FSH beta-subunit, wherein a glycosylation site on the FSH beta-subunit is substituted with an amino acid at which a glycan is not and/or cannot be attached to a subject in need thereof.

The foregoing and other objects and aspects of the present inventive concept are explained in detail in the following set forth below.

### BRIEF DESCRIPTION OF THE DRAWINGS

FIGS. 1A and 1B depict the comparison of recombinant FSH²¹ and fully-glycosylated FSH²⁴ for induction of estradiol and progesterone production in vitro (FIG. TA) and for induction of estradiol production in vivo (FIG. 1B) in Fshb knockout mice.

FIG. 2 depicts the comparison of preantral ovarian follicle induction by recombinant FSH²¹ and fully-glycosylated FSH²⁴.

FIGS. 3A and 3B depict the comparison of bone health histologically (FIG. 3A) and by CT imaging (FIG. 3B) of mice expressing recombinant FSH²¹ and fully-glycosylated FSH²⁴.

FIGS. 4A and 4B depict in vivo dual-energy X-ray absorptiometry (DEXA, or DXA) examining bone mineral density (FIG. 4A) and content (FIG. 4B) of 8-month old female mice expressing FSH¹⁸, FHS²¹, or FSH²⁴ glycoforms.

FIGS. 5A and 5B depict in vivo X-ray computed tomography (CT) analysis of bone density of the femur (FIG. 5A) and tibia (FIG. 5B) of 8-month old female mice expressing FSH¹⁸, FHS²¹, or FSH²⁴ glycoforms.

FIG. 6 depicts in vivo DEXA examining body weight, percent body fat composition, total mass, and total fat tissue mass of 8-month old female mice expressing FSH¹⁸, FHS²¹, or FSH²⁴ glycoforms.

FIG. 7 depicts in vivo MRI analysis examining total body and total fat volume, and abdominal fat percentage of 8-month old female mice expressing FSH¹⁸, FHS²¹, or FSH²⁴ glycoforms.

### DETAILED DESCRIPTION

In the following detailed description, embodiments of the present invention are described in detail to enable practice of the invention. Although the invention is described with reference to these specific embodiments, it should be appreciated that the invention can be embodied in different forms and should not be construed as limited to the embodiments set forth herein. Rather, these embodiments are provided so that this disclosure will be thorough and complete, and will fully convey the scope of the invention to those skilled in the art. All publications cited herein are incorporated by reference in their entireties for their teachings.

The invention includes numerous alternatives, modifications, and equivalents as will become apparent from consideration of the following detailed description.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. Additionally, as used herein, the term “and/or” includes any and all combinations of one or more of the associated listed items and may be abbreviated as “/”.

Unless otherwise defined, all technical terms used herein have the same meaning as commonly understood by one of ordinary skill in the art to which this disclosure belongs.

As used herein, the term “comprise,” in addition to its regular meaning, may also include, and, in some embodiments, may specifically refer to the expressions “consist essentially of” and/or “consist of” Thus, the expression “comprise” can also refer to embodiments, wherein that which is claimed “comprises” specifically listed elements does not include further elements, as well as embodiments wherein that which is claimed “comprises” specifically listed elements may and/or does encompass further elements, or encompass further elements that do not materially affect the basic and novel characteristic(s) of that which is claimed. For example, that which is claimed, such as an amino acid sequence, nucleic acid, nucleic acid sequence, peptide, protein, composition, formulation, cell line, vector, etc. “comprising” specifically listed elements also encompasses, for example, an amino acid sequence, nucleic acid, nucleic acid sequence, peptide, protein, composition, formulation, cell line, vector, etc. “consisting of,” i.e., wherein that which is claimed does not include further elements, and, for example, an amino acid sequence, nucleic acid, nucleic acid sequence, peptide, protein, composition, formulation, cell line, vector, etc. “consisting essentially of,” i.e., wherein that which is claimed may include further elements that do not materially affect the basic and novel characteristic(s) of that which is claimed.

The term “about” generally refers to a range of numeric values that one of skill in the art would consider equivalent to the recited numeric value or having the same function or result. For example, “about” may refer to a range that is within ±1%, ±2%, ±5%, ±10%, ±15%, or even ±20% of the indicated value, depending upon the numeric values that one of skill in the art would consider equivalent to the recited numeric value or having the same function or result. Furthermore, in some embodiments, a numeric value modified by the term “about” may also include a numeric value that is “exactly” the recited numeric value. In addition, any numeric value presented without modification will be appreciated to include numeric values “about” the recited numeric value, as well as include “exactly” the recited numeric value. Similarly, the term “substantially” means largely, but not wholly, the same form, manner or degree and the particular element will have a range of configurations as a person of ordinary skill in the art would consider as having the same function or result. When a particular element is expressed as an approximation by use of the term “substantially,” it will be understood that the particular element forms another embodiment.

As used herein, the terms “treat,” “treating” or “treatment” may refer to any type of action that imparts a modulating effect, which, for example, can be a beneficial and/or therapeutic effect, to a subject afflicted with a condition, disorder, disease or illness, including, for example, improvement in the condition of the subject (e.g., in one or more symptoms), delay in the progression of the disorder, disease or illness, delay of the onset of the disease, disorder, or illness, and/or change in clinical parameters of the condition, disorder, disease or illness, etc., as would be well known in the art.

As used herein, the terms “prevent,” “preventing” or “prevention of” (and grammatical variations thereof) may refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. In representative embodiments, the term “prevent,” “preventing,” or “prevention of” (and grammatical variations thereof) refer to prevention and/or delay of the onset and/or progression of a metabolic disease in the subject, with or without other signs of clinical disease. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

An “effective amount” or “therapeutically effective amount” may refer to an amount of a compound or composition of this invention that is sufficient to produce a desired effect, which can be a therapeutic and/or beneficial effect. The effective amount will vary with the age, general condition of the subject, the severity of the condition being treated, the particular agent administered, during the duration of the treatment, the nature of any concurrent treatment, the pharmaceutically acceptable carrier used, and like factors within the knowledge and expertise of those skilled in the art. As appropriate, an effective amount or therapeutically effective amount in any individual case can be determined by one of ordinary skill in the art by reference to the pertinent texts and literature and/or by using routine experimentation. (See, for example, REMINGTON, THE SCIENCE AND PRACTICE OF PHARMACY (latest edition)).

A “nucleic acid” may include single-stranded and double-stranded nucleic acids and includes ribonucleic acids as well as deoxyribonucleic acids. It may include naturally occurring as well as synthetic nucleotides and can be naturally or synthetically modified.

A “vector” or “expression vector” may refer to and include any intermediary vehicle for a nucleic acid which enables said nucleic acid, for example, to be introduced into prokaryotic and/or eukaryotic host cells and, where appropriate, may be integrated into a genome of and/or expressed in the host cell. Vectors may thus be replicated and/or expressed in the host cells. A vector may include one or more selection markers for selecting host cells comprising the vector. Suitable selection markers include resistance genes which provide the host cell with a resistance IS, e.g. against a specific antibiotic. Further suitable selection markers include, for example, genes for enzymes such as DHFR or GS. Vectors enabling the expression of recombinant proteins including FSH of the inventive concept as described herein, as well as suitable expression cassettes and expression elements which enable the expression of a recombinant protein with high yield in a host cell are well known and are commercially available, and may include any that may be appreciated by one of skill in the art.

A “cell,” “cells,” and a “cell line” may be used interchangeably, and may refer to one or more cells and, in some embodiments, refer to mammalian cells, such as human cells. The term includes progeny of a cell or cell population. One of skill in the art will appreciate that “cells” include progeny of a single cell, and the progeny may not necessarily be completely identical (in morphology or of total DNA complement) to the original parent cell due to natural, accidental, or deliberate mutation and/or change. A “cell” may refer to isolated cells and/or cultivated cells which are not incorporated in a living human, non-human or animal body.

### Compositions

Using Fshb null mice in a pharmacological assay, it has been shown that recombinant FSH²¹ is more bioactive than FSH²⁴ in producing estradiol (Bousfield et al., 2014, Mol. Cell. Endocrinol. 382, 989-997; Wang et al., 2016, Mol. Cell. Endocrinol. 437, 224-236.). Interestingly, commercial preparations of recombinant FSH contain predominantly the FSH²⁴ form. Therefore, using FSH²¹ may be more efficacious and desirable in ART protocols for better follicle induction and estradiol production, which are the 2 most important measures of success of these protocols.

Accordingly, the present inventive concept is based on a recombinant FSH for use in the treatment of metabolic disorders, for example, the treatment of infertility, the treatment and/or prevention of loss of bone density, and/or the treatment and/or prevention of fatty tissue accumulation in a subject in need thereof “FSH” refers to follicle-stimulating hormone, a gonadotropin. In embodiments of the inventive concept, the FSH is human FSH, in particular human FSH, composed of an alpha-subunit and a beta-subunit. “Recombinant FSH,” as used herein, may refer to FSH that is not naturally produced by a living, for example, human or animal body/subject, and then obtained from a sample derived therefrom, such as urine, blood or any other body fluid, waste, e.g., fecal matter, or tissue derived from the human or animal body/subject. In some embodiments, recombinant FSH may be obtained from cells which have been biotechnologically engineered, for example, cells which have been transformed or transfected with a nucleic acid encoding FSH or the alpha- or beta-subunits of FSH. According to some embodiments, recombinant FSH is obtained from human cells, mammalian cells, or non-mammalian, e.g., insect cells, comprising an exogenous nucleic and encoding for an FSH of the inventive concept. Respective exogenous nucleic acids can be introduced e.g., by using one or more expression vectors, for example, an expression vector including a nucleic acid encoding an FSH of the inventive concept as described herein, operably linked to a heterologous promoter, which can be introduced into the host cell e.g., via transfection. Methods for recombinantly producing expression vectors, cell lines including expression vectors, proteins and FSH are well known, and any recombinant method as would be appreciated by one of skill in the art may be used for producing expression vectors, cell lines including expression vectors, proteins and FSH of the present inventive concept. In some embodiments, the recombinant FSH of the inventive concept may be introduced into a host cell by genetic engineering using any method that would be appreciated by one of skill in the art.

As will be appreciated by one of skill in the art, “FSH” is a glycoprotein, i.e., a protein that has been modified by the addition of one or more oligosaccharides, including an alpha-subunit (FSH-α) and a beta-subunit (FSH-β). “Glycosylation” is a process, typically within cells, by which oligosaccharides are covalently attached to, for example, a protein or polypeptide chain. With FSH, glycosylation takes place at particular asparagine (Asn) residues on the alpha-subunit and beta-subunit by N-glycosylation. Fully glycosylated human FSH (FSH²⁴) is a glycoprotein including oligosaccharides, also referred to as glycans or carbohydrates, attached at Asn-52 and Asn-78 of the mature form of FSH-α, and at Asn-7 and Asn-24 of the mature form of FSH-β.

In some embodiments of the present inventive concept, the recombinant FSH is a hypo-glycosylated form of FSH, i.e., an FSH that is not fully glycosylated. In some embodiments, the mature form of FSH-α has, and/or includes, an amino acid sequence as set forth in amino acids 25-116 (SEQ ID NO:1) of GenBank/NCBI Accession No. NP_000726.1 or AAH10957.1, encoded by GenBank/NCBI Accession No. CCDS5007.1, and the mature form of FSH-β has, and/or includes, an amino acid sequence as set forth in amino acids 19-129 (SEQ ID NO:2) of GenBank/NCBI Accession No. NP_000501.1 or AAA52476.1, encoded by GenBank/NCBI Accession No. CCDS 7868.1.

(SEQ ID NO: 1)
APDVQDCPEC TLQENPFFSQ PGAPILQCMG CCFSRAYPTP
LRSKKTMLVQ KNVTSESTCC VAKSYNRVTV MGGFKVENHT
ACHCSTCYYH KS
(SEQ ID NO: 2)
NSCELTNITI AIEKEECRFC ISINTTWCAG YCYTRDLVYK
DPARPKIQKT CTFKELVYET VRVPGCAHHA DSLYTYPVAT
QCHCGKCDSD STDCTVRGLG PSYCSFGEMK E.

In some embodiments, the recombinant FSH-α and FSH-β subunits of the inventive concept may have about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:1 or SEQ ID NO:2, e.g., 1, 1 or 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, or up to 30 amino acid substitutions, additions and/or deletions on one or both subunits, so long as Asn-52 and Asn-78, or their equivalents, of FSH-α, and Asn-7, or its equivalent, of FSH-β are maintained. In some embodiments, the recombinant FSH-α and FSH-β subunits of the inventive concept may include 1 or 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, or up to 30 amino acid additions, for example, amino acid additions to the N-terminal and/or the C-terminal of a recombinant FSH-α and FSH-β subunit comprising the sequence of SEQ ID NO:1 or SEQ ID NO:2, or may include 1 or 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, or up to 30 amino acid additions, for example, amino acid additions to the N-terminal and/or the C-terminal of a FSH-β subunit including a sequence having 90%, 91%, 92%, 93%, 94%, 95% 96%, 97%, 98%, or 99% sequence identity to SEQ ID NO:2. In some embodiments of the present inventive concept, the recombinant hypo-glycosylated form of FSH includes glycans attached at Asn-52 and Asn-78 of the mature form of FSH-α, for example, as set forth in SEQ ID NO:1, and a glycan attached at Asn-7 of the mature form of FSH-β, for example, as set forth in SEQ ID NO:2. This hypo-glycosylated form of FSH is also known as FSH²¹, and is not glycosylated at Asn-24. In other embodiments, hypo-glycosylated form of FSH includes glycans attached at Asn-52 and Asn-78 of the mature form of FSH-α, for example, as set forth in SEQ ID NO:1, and a glycan attached at Asn-24 of the mature form of FSH-β, for example, as set forth in SEQ ID NO:2. This hypo-glycosylated form of FSH is also known as FSH¹⁸, and is not glycosylated at Asn-7.

In some embodiments of the present inventive concept, the recombinant hypo-glycosylated form of FSH includes a mature form of FSH-β having and/or including an amino acid sequence as set forth in SEQ ID NO:3, wherein Asn-24 of the mature form of FSH-β (Asn-42 of GenBank/NCBI Accession No. NP_000501.1 or AAA52476.1) is replaced with an Alanine (Ala, N24A), or having and/or including an amino acid sequence as set forth in SEQ ID NO:4, wherein Asn-24 of the mature form of FSH-β is replaced with an Glutamine (Gln, N24Q), either of which amino acid substitution replaces the N-glycosylation site at amino acid position 24 (Asn-24) of these modified forms of the FSH beta-subunit with an amino acid that is not naturally glycosylated, i.e., an amino acid at which a glycan is not and/or cannot be attached through any of the typical glycosylation processes that naturally occur, e.g., N-glycosylation, O-glycosylation, phosphoserine glycosylation, C-mannosylation, and glypiation. As such, since N-glycosylation only takes place at Asn-7 and does not take place at amino acid position 24 of these modified recombinant FSH-β subunits, only FSH²¹ is produced by systems/cells expressing these modified recombinant FSH-β subunits. In some embodiments, the mature form of the modified recombinant FSH-β is encoded for by, for example, nucleotides 55-387 of the sequence as set forth in SEQ ID NO:5. In some embodiments, the modified recombinant FSH-β of the inventive concept is encoded by, for example, a nucleic acid including the sequence as set forth in SEQ ID NO:5. In some embodiments, the mature form of the modified recombinant FSH-β is encoded by nucleotides 55-387 of SEQ ID NO:5. The nucleic acid may further include a heterologous promoter. In some embodiments, the nucleic acid may be part of a vector or expression vector including the sequence as set forth by nucleotides 55-387 of SEQ ID NO:5, i.e., the nucleic acid sequence as set forth in SEQ ID NO:6. In some embodiments, the nucleic acid may be part of a vector or expression vector including the sequence as set forth in SEQ ID NO:5.

(SEQ ID NO: 3)
NSCELTNITI AIEKEECRFC ISIATTWCAG YCYTRDLVYK
DPARPKIQKT CTFKELVYET VRVPGCAHHA DSLYTYPVAT
QCHCGKCDSD STDCTVRGLG PSYCSFGEMK E
(SEQ ID NO: 4)
NSCELTNITI AIEKEECRFC ISIQTTWCAG YCYTRDLVYK
DPARPKIQKT CTFKELVYET VRVPGCAHHA DSLYTYPVAT
QCHCGKCDSD STDCTVRGLG PSYCSFGEMK E.
(SEQ ID NO: 5)
ATG AAG ACA CTC CAG TTT TTC TTC CTT TTC TGT
TGC TGG AAA GCA ATC TGC TGC AAT AGC TGT GAG
CTG ACC AAC ATC ACC ATT GCA ATA GAG AAA GAA
GAA TGT CGT TTC TGC ATA AGC ATC CAG ACC ACT
TGG TGT GCT GGC TAC TGC TAC ACC AGG GAT CTG
GTG TAT AAG GAC CCA GCC AGG CCC AAA ATC CAG
AAA ACA TGT ACC TTC AAG GAA CTG GTA TAC GAA
ACA GTG AGA GTG CCC GGC TGT GCT CAC CAT GCA
GAT TCC TTG TAT ACA TAC CCA GTG GCC ACC CAG
TGT CAC TGT GGC AAG TGT GAC AGC GAC AGC ACT
GAT TGT ACT GTG CGA GGC CTG GGG CCC AGC TAC
TGC TCC TTT GGT GAA ATG AAA GAA TAA
(SEQ ID NO: 6)
AAT AGC TGT GAG CTG ACC AAC ATC ACC ATT GCA
ATA GAG AAA GAA GAA TGT CGT TTC TGC ATA AGC
ATC CAG ACC ACT TGG TGT GCT GGC TAC TGC TAC
ACC AGG GAT CTG GTG TAT AAG GAC CCA GCC AGG
CCC AAA ATC CAG AAA ACA TGT ACC TTC AAG GAA
CTG GTA TAC GAA ACA GTG AGA GTG CCC GGC TGT
GCT CAC CAT GCA GAT TCC TTG TAT ACA TAC CCA
GTG GCC ACC CAG TGT CAC TGT GGC AAG TGT GAC
AGC GAC AGC ACT GAT TGT ACT GTG CGA GGC CTG
GGG CCC AGC TAC TGC TCC TTT GGT GAA ATG AAA
GAA.

In some embodiments, the recombinant FSH-β subunit of the inventive concept may have about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:3 or SEQ ID NO:4, e.g., 1, 1 or 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, or up to 30 amino acid substitutions, additions and/or deletions on one or both subunits, so long as Asn-7, or its equivalent, of FSH-β are maintained, and Ala-24, or its equivalent, of SEQ ID NO:3, or Gln-24, or its equivalent, of SEQ ID NO:4, is not replaced with an amino acid that may be glycosylated. In some embodiments, the recombinant FSH-β subunit of the inventive concept, encoded by, for example, a nucleic acid including a sequence as set forth by SEQ ID NO:5 or SEQ ID NO:6, may include 1 or 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, or up to 30 amino acid additions, for example, amino acid additions to the N-terminal and/or the C-terminal of a recombinant FSH-β subunit including a sequence of SEQ ID NO:3 or SEQ ID NO:4, or may include 1 or 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, or up to 30 amino acid additions, for example, amino acid additions to the N-terminal and/or the C-terminal of a recombinant FSH-β subunit including a sequence having about 90%, 91%, 92%, 93%, 94%, 95%, 96%, 97%, 98%, or 99% sequence identity to SEQ ID NO:3 or SEQ ID NO:4.

In some embodiments, the recombinant FSH-β subunit including a sequence having about 90%, 91%, 92%, 93%, 94%, 95%, 96%, 97%, 98%, or 99% sequence identity to SEQ ID NO:3 or SEQ ID NO:4 may have 1-30 amino acid additions to the N-terminal and/or C-terminal. In some embodiments, the amino acid additions may be at the C-terminal of the recombinant FSH-α and/or FSH-β subunit. In some embodiments, the amino acid additions may be included/added to the C-terminal of the recombinant FSH-β subunit. In some embodiments, the amino acid additions may include sequences from, for example, a subunit of a gonadotropin hormone, such as a beta subunit from a gonadotropin hormone. Gonadotropin hormones include, for example, luteinizing hormone (LH), thyroid-stimulating hormone (TSH), chorionic gonadotropin (CG), as well as FSH. In some embodiments, amino acid sequences included as additions to the recombinant FSH-beta subunit of the inventive concept are from a chorionic gonadotropin-beta subunit, for example, a human chorionic gonadotropin-beta subunit (hCG-β) as set forth in, for example, GenBank Accession No. AAA53287.1, and NCBI reference sequences NP_000728.1, NP_001305994.1, NP_203696.2, and NP_001372190.1. In some embodiments, the amino acid additions to the C-terminal of a recombinant FSH-β subunit of the inventive concept may include, for example, an amino acid sequence from the hCG-β subunit as set forth in SEQ ID NO:11, found at the C-terminal of the hCG-β subunit.

(SEQ ID NO: 11)
SSSSKAPPPS LPSPSRLPGP SDTPILPQ.

Accordingly, in some embodiments, the recombinant FSH-β subunit of the inventive concept, including amino acid additions to the C-terminal, i.e., including a C-terminal peptide (CTP) addition, may include a sequence as set forth in SEQ ID NO:12, and may be encoded by a nucleic acid sequence including, for example, the sequences as set forth in SEQ ID NO:13 and/or SEQ ID NO:14.

(SEQ ID NO: 12)
NSCELTNITI AIEKEECRFC ISIQTTWCAG YCYTRDLVYK
DPARPKIQKT CTFKELVYET VRVPGCAHHA DSLYTYPVAT
QCHCGKCDSD STDCTVRGLG PSYCSFGEMK ESSSSKAPPP
SLPSPSRLPG PSDTPILPQ
(SEQ ID NO: 13)
ATG AAG ACA CTC CAG TTT TTC TTC CTT TTC TGT TGC
TGG AAA GCA ATC TGC TGC AAT AGC TGT GAG CTG ACC
AAC ATC ACC ATT GCA ATA GAG AAA GAA GAA TGT CGT
TTC TGC ATA AGC ATC CAG ACC ACT TGG TGT GCT GGC
TAC TGC TAC ACC AGG GAT CTG GTG TAT AAG GAC CCA
GCC AGG CCC AAA ATC CAG AAA ACA TGT ACC TTC AAG
GAA CTG GTA TAC GAA ACA GTG AGA GTG CCC GGC TGT
GCT CAC CAT GCA GAT TCC TTG TAT ACA TAC CCA GTG
GCC ACC CAG TGT CAC TGT GGC AAG TGT GAC AGC GAC
AGC ACT GAT TGT ACT GTG CGA GGC CTG GGG CCC AGC
TAC TGC TCC TTT GGT GAA ATG AAA GAA TCC TCT TCC
TCA AAG GCC CCT CCC CCC AGC CTT CCA AGC CCA TCC
CGA CTC CCG GGG CCC TCG GAC ACC CCG ATC CTC CCA
CAA
(SEQ ID NO: 14)
AAT AGC TGT GAG CTG ACC AAC ATC ACC ATT GCA ATA
GAG AAA GAA GAA TGT CGT TTC TGC ATA AGC ATC CAG
ACC ACT TGG TGT GCT GGC TAC TGC TAC ACC AGG GAT
CTG GTG TAT AAG GAC CCA GCC AGG CCC AAA ATC CAG
AAA ACA TGT ACC TTC AAG GAA CTG GTA TAC GAA ACA
GTG AGA GTG CCC GGC TGT GCT CAC CAT GCA GAT TCC
TTG TAT ACA TAC CCA GTG GCC ACC CAG TGT CAC TGT
GGC AAG TGT GAC AGC GAC AGC ACT GAT TGT ACT GTG
CGA GGC CTG GGG CCC AGC TAC TGC TCC TTT GGT GAA
ATG AAA GAA TCC TCT TCC TCA AAG GCC CCT CCC CCC
AGC CTT CCA AGC CCA TCC CGA CTC CCG GGG CCC TCG
GAC ACC CCG ATC CTC CCA CAA.

In other embodiments of the present inventive concept, the recombinant hypo-glycosylated form of FSH includes a mature form of FSH-β having an amino acid sequence as set forth in SEQ ID NO:7, wherein Asn-7 of the mature form of FSH-β (Asn-25 of GenBank/NCBI Accession No. NP_000501.1 or AAA52476.1) is replaced with an Alanine (Ala, N7A), or having an amino acid sequence as set forth in SEQ ID NO:8, wherein Asn-7 of the mature form of FSH-β is replaced with an Glutamine (Gln, N7Q), either of which amino acid substitution replaces the N-glycosylation site at amino acid position 7 (Asn-7) of these modified forms of the FSH beta-subunit with an amino acid that is not naturally glycosylated, i.e., an amino acid at which a glycan is not and/or cannot be attached through any of the typical glycosylation processes that naturally occur, e.g., N-glycosylation, O-glycosylation, phosphoserine glycosylation, C-mannosylation, and glypiation. As such, since N-glycosylation only takes place at Asn-24 and does not take place at amino acid position 7 of these modified recombinant FSH-β subunits, only FSH¹⁸ is produced by systems/cells expressing these modified recombinant FSH-β subunits. In some embodiments, the modified recombinant FSH-β of the inventive concept is encoded by, for example, a nucleic acid including the sequence as set forth in SEQ ID NO:9. In some embodiments, the mature form of the modified recombinant FSH-β is encoded by nucleotides 55-387 of SEQ ID NO:9. The nucleic acid may further include a heterologous promoter. In some embodiments, the nucleic acid may be part of a vector or expression vector including the sequence as set forth by nucleotides 55-387 of SEQ ID NO:9, i.e., the nucleic acid sequence as set forth in SEQ ID NO:10. In some embodiments, the nucleic acid may be part of a vector or expression vector including the sequence as set forth in SEQ ID NO:9.

(SEQ ID NO: 7)
NSCELTAITI AIEKEECRFC ISINTTWCAG YCYTRDLVYK
DPARPKIQKT CTFKELVYET VRVPGCAHHA DSLYTYPVAT
QCHCGKCDSD STDCTVRGLG PSYCSFGEMK E
(SEQ ID NO : 8)
NSCELTQITI AIEKEECRFC ISINTTWCAG YCYTRDLVYK
DPARPKIQKT CTFKELVYET VRVPGCAHHA DSLYTYPVAT
QCHCGKCDSD STDCTVRGLG PSYCSFGEMK E.
(SEQ ID NO: 9)
ATG AAG ACA CTC CAG TTT TTC TTC CTT TTC TGT TGC
TGG AAA GCA ATC TGC TGC AAT AGC TGT GAG CTG ACC
CAG ATC ACC ATT GCA ATA GAG AAA GAA GAA TGT CGT
TTC TGC ATA AGC ATC AAC ACC ACT TGG TGT GCT GGC
TAC TGC TAC ACC AGG GAT CTG GTG TAT AAG GAC CCA
GCC AGG CCC AAA ATC CAG AAA ACA TGT ACC TTC AAG
GAA CTG GTA TAC GAA ACA GTG AGA GTG CCC GGC TGT
GCT CAC CAT GCA GAT TCC TTG TAT ACA TAC CCA GTG
GCC ACC CAG TGT CAC TGT GGC AAG TGT GAC AGC GAC
AGC ACT GAT TGT ACT GTG CGA GGC CTG GGG CCC AGC
TAC TGC TCC TTT GGT GAA ATG AAA GAA
(SEQ ID NO: 10)
AAT AGC TGT GAG CTG ACC CAG ATC ACC ATT GCA ATA
GAG AAA GAA GAA TGT CGT TTC TGC ATA AGC ATC AAC
ACC ACT TGG TGT GCT GGC TAC TGC TAC ACC AGG GAT
CTG GTG TAT AAG GAC CCA GCC AGG CCC AAA ATC CAG
AAA ACA TGT ACC TTC AAG GAA CTG GTA TAC GAA ACA
GTG AGA GTG CCC GGC TGT GCT CAC CAT GCA GAT TCC
TTG TAT ACA TAC CCA GTG GCC ACC CAG TGT CAC TGT
GGC AAG TGT GAC AGC GAC AGC ACT GAT TGT ACT GTG
CGA GGC CTG GGG CCC AGC TAC TGC TCC TTT GGT GAA
ATG AAA GAA

In some embodiments, the recombinant FSH-β subunit of the inventive concept may have about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:7 or SEQ ID NO:8, may include e.g., 1 or 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, or up to 30 amino acid substitutions, additions and/or deletions on one or both subunits, so long as Asn-24, or its equivalent, of FSH-β are maintained, and Ala-7, or its equivalent, of SEQ ID NO:7, or Gln-7, or its equivalent, of SEQ ID NO:8, is not replaced with an amino acid that may be glycosylated. In some embodiments, the recombinant FSH-α and FSH-β subunits of the inventive concept, encoded by, for example, a nucleic acid including a sequence as set forth by SEQ ID NO:9 or SEQ ID NO:10, may include 1 or 2, up to 3, up to 4, up to 5, up to 6, up to 7, up to 8, up to 9, up to 10, up to 11, up to 12, up to 13, up to 14, up to 15, up to 16, up to 17, up to 18, up to 19, or up to 20 amino acid additions, for example, amino acid additions to the N-terminal and/or the C-terminal of a recombinant FSH-α and FSH-β subunit including the sequence of SEQ ID NO:7 or SEQ ID NO:8, or a recombinant FSH-α and FSH-β subunit may include 1 or 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, or up to 30 amino acid additions, for example, amino acid additions to the N-terminal and/or the C-terminal of a sequence having 90%, 91%, 92%, 93%, 94%, 95%, 96%, 97%, 98%, or 99% sequence identity to SEQ ID NO:7 or SEQ ID NO:8. In some embodiments, the recombinant FSH-β subunit including a sequence having about 90%, 91%, 92%, 93%, 94%, 95%, 96%, 97%, 98%, or 99% sequence identity to SEQ ID NO:7 or SEQ ID NO:8 may have 1-30 amino acid additions to the N-terminal and/or C-terminal. In some embodiments, the amino acid additions may be at the C-terminal of the recombinant FSH-α and/or FSH-β subunit. In some embodiments, the amino acid additions may be included/added to the C-terminal of the recombinant FSH-β subunit. In some embodiments, the amino acid additions may include sequences from, for example, a subunit of a gonadotropin hormone, such as a beta subunit from a gonadotropin hormone. Gonadotropin hormones include, for example, luteinizing hormone (LH), thyroid-stimulating hormone (TSH), chorionic gonadotropin (CG), as well as FSH. In some embodiments, amino acid sequences included as additions to the recombinant FSH-beta subunit of the inventive concept are from a chorionic gonadotropin-beta subunit, for example, a human chorionic gonadotropin-beta subunit (hCG-β) as set forth in, for example, GenBank Accession No. AAA53287.1, and NCBI reference sequences NP_000728.1, NP_001305994.1, NP_203696.2, and NP_001372190.1. In some embodiments, the amino acid additions to the C-terminal of a recombinant FSH-β subunit of the inventive concept may include, for example, an amino acid sequence from the hCG-D subunit as set forth in SEQ ID NO:11, found at the C-terminal of the hCG-D subunit. Accordingly, in some embodiments, the recombinant FSH-β subunit, including amino acid additions to the C-terminal, i.e., including a C-terminal peptide (CTP) addition, may include a sequence as set forth in SEQ ID NO:15, and may be encoded by a nucleic acid sequence including, for example, the sequences as set forth in SEQ ID NO:16 and/or SEQ ID NO:17.

(SEQ ID NO: 15)
NSCELTQITI AIEKEECRFC ISINTTWCAG YCYTRDLVYK
DPARPKIQKT CTFKELVYET VRVPGCAHHA DSLYTYPVAT
QCHCGKCDSD STDCTVRGLG PSYCSFGEMK ESSSSKAPPP
SLPSPSRLPG PSDTPILPQ
(SEQ ID NO: 16)
ATG AAG ACA CTC CAG TTT TTC TTC CTT TTC TGT TGC
TGG AAA GCA ATC TGC TGC AAT AGC TGT GAG CTG ACC
CAG ATC ACC ATT GCA ATA GAG AAA GAA GAA TGT CGT
TTC TGC ATA AGC ATC AAC ACC ACT TGG TGT GCT GGC
TAC TGC TAC ACC AGG GAT CTG GTG TAT AAG GAC CCA
GCC AGG CCC AAA ATC CAG AAA ACA TGT ACC TTC AAG
GAA CTG GTA TAC GAA ACA GTG AGA GTG CCC GGC TGT
GCT CAC CAT GCA GAT TCC TTG TAT ACA TAC CCA GTG
GCC ACC CAG TGT CAC TGT GGC AAG TGT GAC AGC GAC
AGC ACT GAT TGT ACT GTG CGA GGC CTG GGG CCC AGC
TAC TGC TCC TTT GGT GAA ATG AAA GAA TCC TCT TCC
TCA AAG GCC CCT CCC CCC AGC CTT CCA AGC CCA TCC
CGA CTC CCG GGG CCC TCG GAC ACC CCG ATC CTC CCA
CAA
(SEQ ID NO: 17)
AAT AGC TGT GAG CTG ACC CAG ATC ACC ATT GCA ATA
GAG AAA GAA GAA TGT CGT TTC TGC ATA AGC ATC AAC
ACC ACT TGG TGT GCT GGC TAC TGC TAC ACC AGG GAT
CTG GTG TAT AAG GAC CCA GCC AGG CCC AAA ATC CAG
AAA ACA TGT ACC TTC AAG GAA CTG GTA TAC GAA ACA
GTG AGA GTG CCC GGC TGT GCT CAC CAT GCA GAT TCC
TTG TAT ACA TAC CCA GTG GCC ACC CAG TGT CAC TGT
GGC AAG TGT GAC AGC GAC AGC ACT GAT TGT ACT GTG
CGA GGC CTG GGG CCC AGC TAC TGC TCC TTT GGT GAA
ATG AAA GAA TCC TCT TCC TCA AAG GCC CCT CCC CCC
AGC CTT CCA AGC CCA TCC CGA CTC CCG GGG CCC TCG
GAC ACC CCG ATC CTC CCA CAA.

Preparation of the recombinant hypo-glycosylated form of FSH include any recombinant methods that would be appreciated by one of skill in the art. See, e.g., SAMBROOK et al. MOLECULAR CLONING: A LABORATORY MANUAL 2nd Ed. (Cold Spring Harbor, N.Y., 1989); AUSUBEL et al. CURRENT PROTOCOLS IN MOLECULAR BIOLOGY (Green Publishing Associates, Inc. and John Wiley & Sons, Inc., New York). Although the recombinant hypo-glycosylated form of FSH may be produced in any cell line, in some embodiments, FSH, recombinant FSH and/or recombinant hypo-glycosylated FSH of the inventive concept is a human FSH is obtained/derived from human or mammalian cells, and in some embodiments, obtained/derived from a human or mammalian cell line. Human cell lines used to produce recombinant FSH of the inventive concept may include, for example, GT-5s, or Per.C6. In some embodiments, recombinant FSH, such as the hypo-glycosylated form of FSH of the inventive concept, is produced in a cell line derived from the pituitary gland, such as the cell line GH3, derived from a rat pituitary tumor. In other embodiments, the recombinant FSH of the inventive concept may be expressed and produced and isolated from non-mammalian cell line, for example, insect cells in which a baculovirus expression vector system may be used, such as, but not limited to, for example, BTI-Tn-5B1-4 (High Five or Hi5) cells.

It will also be appreciated that glycan structures that may be included at the glycosylation sites on the FSH and recombinant FSH of the inventive concept are not particularly limited. Sugars that are part of the glycan structure may include, for example, fructose, galactose, mannose, galactosamine, glucosamine, and/or sialic acid. Nonetheless, in some embodiments, it will be appreciated that the FSH and recombinant FSH of the inventive concept will include glycan structures, for example, a glycan/sugar structure at Asn-52 and/or Asn-78 of human FSH-α, or their equivalents, and a glycan/sugar structure at Asn-7 and/or at Asn-24 of human FSH-β, or their equivalents, that will be identical to or substantially identical to the glycan structures of FSH produced in the pituitary gland of a human or mammalian body/subject.

According to further embodiments of the present inventive concept, provided are pharmaceutical compositions including a therapeutically effective amount of the recombinant hypo-glycosylated form of FSH. In some embodiments, the pharmaceutical composition further comprises a pharmaceutically acceptable carrier. The term “pharmaceutically acceptable carrier” as used herein refers to any substance, not itself a therapeutic agent, used as at least in part a vehicle for delivery of a therapeutic agent to a subject. Non-limiting examples of pharmaceutically acceptable components include, without limitation, any of the standard pharmaceutical carriers such as phosphate buffered saline solutions, water, emulsions such as oil/water emulsions or water/oil emulsions, microemulsions, and various types of wetting agents. Further, in preparing such pharmaceutical compositions comprising the active ingredient or ingredients in admixture with components necessary for the formulation of the compositions, other conventional pharmacologically acceptable additives may be incorporated, for example, excipients, stabilizers, wetting agents, emulsifying agents, lubricants, sweetening agents, coloring agents, flavoring agents, isotonicity agents, buffering agents, antioxidants and the like. Additives may include, for example, starch, mannitol, sorbitol, precipitated calcium carbonate, crystalline cellulose, carboxymethylcellulose, dextrin, gelatin, acacia, EDTA, magnesium stearate, talc, hydroxypropylmethylcellulose, sodium metabisulfite, and the like.

Formulations suitable for administering the composition of the present inventive concept may be suitable for any route of administration as would be appreciated by one of skill in the art. Routes of administration may include, but are not limited to intravenous, oral, parenteral, subcutaneous, topical and/or vaginal methods of administration.

### Methods of Administration

Another embodiment of the present inventive concept provides a method for administering to a subject in need thereof a compound or pharmaceutical composition as described herein. For administration, either the compound or pharmaceutical composition is understood as being the active ingredient and capable of administration to a subject, and thus, in some instances, the terms are interchangeable.

The method of administration of the compound or pharmaceutical composition as described herein is not particularly limited, and any method that would be appreciated by one of skill in the art for the compound or pharmaceutical composition in a particular formulation as described herein. Routes of administration may include, but are not limited to intravenous, oral, parenteral, subcutaneous, topical and/or vaginal methods of administration. In some embodiments, methods of administration may include utilization of a minipump or a micropump for delivery of the compound or pharmaceutical composition as described herein.

Subjects suitable to be treated with the composition, compositions and formulations of the present invention include, but are not limited to mammalian subjects. Mammals according to the present invention include, but are not limited to, canines, felines, bovines, caprines, equines, ovines, porcines, rodents (e.g., rats and mice), lagomorphs, primates, humans and the like, and mammals in utero. Any mammalian subject in need of being treated or desiring treatment according to the present invention is suitable. In some embodiments of the present inventive concept, the subject is a human subject. Although the human subject treated according to methods of the present inventive concept may be of any gender (for example, male, female or transgender) and at any stage of development (i.e., neonate, infant, juvenile, adolescent, adult, elderly). In some embodiments, the human subject is a premenopausal, menopausal, or postmenopausal female human subject. In other embodiments of the present inventive concept, the subject may be a larger mammalian subject, such as a bovine subject, an ovine subject, or a non-human primate.

Dosages of compositions and formulations of the present inventive concept may depend on route of administration and intended use. It will be appreciated by one of skill in the art that international units (IU) for FSH may refer to the fourth International Standard for human urinary FSH (Stoning and Gaines Das 2001, J. Endocrinol. 171, 119-129), determined as described by Steelman and Pohley 1953, Endocrinol. 53, 604-616. For example, 450 IU of FSH, corresponds to 33 mcg of FSH (41 IU corresponds to 3 mcg, ˜13.67 IU/mcg). Typical dosages available of commercially available FSH, such as GONAL-F®, GONAL-F RFF®, FOLLISTIM AQ®, and PUREGON®, may be in a range of about 1-10,000 IU, 10-3,600 IU and/or 37.5-1,200 IU, and include dosages of, for example, 37.5 IU, 75 IU, 150 IU, 300 IU, 450 IU, 600 IU, 750 IU, 900 IU, 1,050 IU, and 1,200 IU, which may be provided as a solid for dissolving in a carrier for administration, such as subcutaneous administration, or provided as a solution in, for example, 0.75 mL or 1.0 mL volume of a suitable carrier for subcutaneous administration/injection. Dosages and dosing regimens of FSH compositions are known, and any dosage and dosing regimens for FSH compositions and formulations may be used for the FSH compositions and formulations of the present inventive concept. Known dosing regimens include, for example, daily subcutaneous administration, initially for 5 days, 7 days or 14 days, with duration of therapy running until response is achieved.

### FSH Agonists

In some embodiments of the present inventive concept, provided are FSH agonists, compositions comprising FSH agonists, and methods of using FSH agonists. Any agent that has FSH agonist activity, including agents with enhanced activities associated with FSH, may be considered within the scope of the inventive concept, for example, but not limited to, protein- or peptide-based FSH agonists. Embodiments of FSH agonists of the inventive concept include modified/recombinant forms of FSH that activate and/or enhance activities/bioactivities associated with FSH binding to the FSH receptor (FSHR). In some embodiments, FSH agonists of the inventive concept include modified/recombinant forms of FSH, for example, forms of FSH in which N-linked glycosylation cannot take place, for example, a recombinant FSH subunit wherein a glycosylation site on the beta-subunit is substituted with an amino acid at which a glycan is not and/or cannot be attached. It will be appreciated by one of skill in the art that the tripeptide Asn-X-Ser/Thr is required for glycosylation to take place at the Asn residue of the tripeptide. In some embodiments, FSH agonists include a recombinant FSH-beta subunit including an amino acid substitution at Asn-7 and/or at Asn-24, or their equivalents, wherein the amino acid substitution is not and/or cannot be glycosylated. In some embodiments, the amino acid substitution may be glutamine (Q) for Asn (N). In some embodiments, the amino acid substitution may be alanine (A) for N. Nevertheless, any amino acid substitution, such as, but not limited to, an amino acid substitution at Asn-7 and/or Asn-24, so long as glycosylation does not/cannot take place at amino acid 7 and/or amino acid 24, or their equivalents, and activity/bioactivity associated with the FSHR binding of the FSH agonist takes place, and/or activity/bioactivity associated with FSHR binding of the FSH agonist is enhanced.

In some embodiments, the FSH agonist of the inventive concept includes a recombinant FSH subunit that may have 1-30 amino acid additions to the N-terminal and/or C-terminal. In some embodiments, the amino acid additions may be at the C-terminal of the recombinant FSH-alpha and/or recombinant FSH-beta subunit. In some embodiments, the amino acid additions may be included/added to the C-terminal of the recombinant FSH-beta subunit. In some embodiments, the amino acid additions may include sequences from, for example, a subunit of a gonadotropin hormone, such as a beta subunit from a gonadotropin hormone. Gonadotropin hormones include, for example, luteinizing hormone (LH), thyroid-stimulating hormone (TSH), chorionic gonadotropin (CG), as well as FSH. In some embodiments, amino acid sequences included as additions to the recombinant FSH-beta subunit of the inventive concept are from a chorionic gonadotropin-beta subunit, for example, a human chorionic gonadotropin-beta subunit (hCG-β) as set forth in, for example, an amino acid sequence from the hCG-β subunit as set forth in SEQ ID NO:11, found at the C-terminal of the hCG-β subunit. Accordingly, in some embodiments, FSH agonists of the inventive concept include a recombinant FSH-beta subunit, including amino acid additions to the C-terminal, i.e., including a C-terminal peptide (CTP) addition.

Activities/bioactivities, and enhanced activities/bioactivities, associated with binding of FSH agonists of the inventive concept to the FSHR include, for example, uses of compounds, compositions, and formulations, such as are described below.

### USES

Compounds, compositions, and formulations for use, and methods of use of compounds, compositions, and formulations of the present inventive concept include, for example, compounds, compositions, and formulations of the inventive concept, including FSH agonists of the inventive concept for use in, for methods of use in, for use in the manufacture of a medicament for, and for methods of, treatment and/or prevention of a number of medical conditions, including, for example, methods of treating infertility, methods of inducing oocyte/egg growth and/or maturation, methods of stimulating sex steroid secretion, methods for treating and/or preventing the loss of bone density, for example, such as the loss of bone density as a result of osteoporosis, and methods for treating and/or preventing fatty tissue accumulation, for example, such as fatty tissue accumulation associated with the onset of menopause, in a subject in need thereof.

Treating infertility, or infertility treatment, according to embodiments of the inventive concept include treatment of a dysfunction, disorder, or disease related to reproduction or fertility in a human or non-human (animal) subject including administration of compositions and/or formulations of the inventive concept as described herein. In some embodiments, treating infertility includes assisted reproductive technologies (ART), including but not limited to in vitro fertilization, in vitro fertilization with intracytoplasmic sperm injection, ovulation induction, and ovulation induction with intrauterine insemination. In some embodiments, treating infertility includes treatment of ovulatory disorders, treatment of disorders related to egg maturation, as well as inducing, enabling and/or improving folliculogenesis, inducing, enabling and/or improving spermatogenesis, inducing, enabling and/or improving Sertoli cell proliferation, and treating hypogonadotropic hypogonadism in the subject.

In some embodiments, treating infertility according to the inventive concept may include methods of inducing and/or stimulating secretion/production of sex steroids in the subject including administration of compositions and/or formulations of the inventive concept as described herein. Sex steroids, or sex hormones, refer to steroid hormones that interact with androgen or estrogen receptors. Sex steroids include: androgens, e.g., anabolic steroids, androstenedione, dehydroepiandrosterone, dihydrotestosterone and testosterone; estrogens, e.g., estradiol, estriol and estrone; and progesterone. In some embodiments, inducing and/or stimulating secretion/production of sex steroids include inducing and/or stimulating secretion/production of estrogens, for example, estradiol, and/or inducing and/or stimulating secretion/production progesterone.

In some embodiments, treating infertility according to the inventive concept may include induction of follicle growth and/or maturation including administration of compositions and/or formulations of the inventive concept as described herein. In some embodiments, treating infertility may include induction of oocyte/egg growth and/or maturation including administration of compositions and/or formulations of the inventive concept as described herein.

In some embodiments, treating infertility according to the inventive concept may include inducing, enabling and/or improving Sertoli cell proliferation including administration of compositions and/or formulations of the inventive concept as described herein. In some embodiments, treating infertility may include treating hypogonadotropic hypogonadism including administration of compositions and/or formulations of the inventive concept as described herein.

Treating and/or preventing loss of bone density according to embodiments of the inventive concept may include treating and/or preventing conditions and diseases including administration of compositions and/or formulations of the inventive concept as described herein to a subject in which the inhibition of bone loss and/or the promotion of bone formation is desirable. Such conditions and diseases include, for example, osteoporosis, osteomyelitis, Paget's disease, periodontitis, hypercalcemia, osteonecrosis, osteosarcoma, osteolyic metastases, familial expansile osteolysis, prosthetic loosening, periprostetic osteolysis, cleiodocranial dysplasia (CCD), osteoporosis from arthritides, and bone loss due to metastatic disease and humoral hypercalcemia. In some embodiments, treating and/or preventing loss of bone density includes methods of treating and/or preventing osteoporosis in a subject. In some embodiments, treating and/or preventing loss of bone density includes treating a human subject. In some embodiments, the human subject is a female human subject. In some embodiments, the female human subject. In some embodiments, the female human subject may be premenopausal. In some embodiments, the female human subject may be perimenopausal. In some embodiments, the female subject may be postmenopausal, or in menopause.

Treating and/or preventing fatty tissue accumulation according to embodiments of the inventive concept may include administration of compositions and/or formulations of the inventive concept as described herein to treat and/or prevent fatty tissue accumulation that may result from, for example, a female subject, such as a female human subject approaching or entering menopause, e.g., a perimenopausal female human subject, or treating and/or preventing fatty tissue accumulation in a female human subject that is in menopause, but is not limited thereto.

The present invention is more particularly described in the following examples, which is intended to be illustrative only, since numerous modifications and variations therein will be apparent to those skilled in the art.

### Examples

### 1. Recombinant Hypo-Glycosylated FSH²¹

Hypo-glycosylated FSH²¹ contains one N-linked sugar chain attached to the Asn⁷ residue of the human FSH-β subunit. Current commercially available FSH preparations include predominantly fully glycosylated FSH²⁴, in which glycan structures are present on both Asn⁷ and Asn²⁴ residues of the FSH-β subunit. The product developed is a recombinant human FSH²¹ expressed in GH3 cells derived from a rat pituitary gland tumor. The recombinant FSH²¹ produced in GH3 cells has been modified by site-directed mutagenesis, substituting Asn²⁴ with Alanine, thus eliminating the Asn²⁴ glycosylation site, and as such the recombinant FSH produced in this GH3 cell genomic clone is a recombinant FSH²¹, glycosylated at Asn⁵² and Asn⁷⁸ of FSH-α, and only at Asn⁷ of FSH-β.

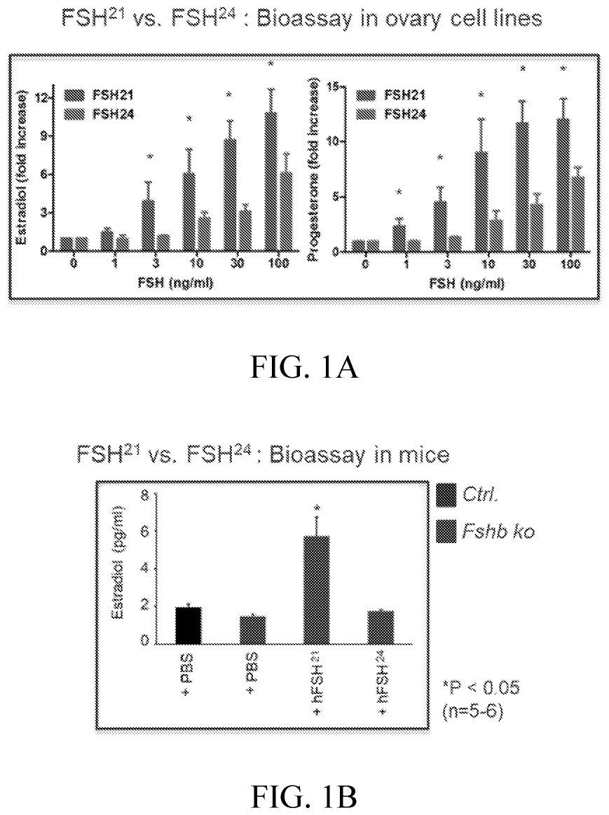

Induction of estradiol and progesterone production by this recombinant FSH²¹ was compared to FSH²⁴ in vitro in a human ovary KGN granulosa cell line, and induction of estradiol production by this recombinant FSH²¹ was compared to FSH²⁴ in vivo by administration to Fshb knockout mice. The results of this examination are shown in FIGS. 1A and 1B. The in vitro studies show that this recombinant FSH²¹ is more effective at inducing both estradiol and progesterone production than full-glycosylated FSH²⁴ at all FSH concentrations from 1-100 ng/ml (FIG. 1A). The in vivo studies show administration of this recombinant FSH²¹ is superior to administration of fully-gly cosylated FSH²⁴ at inducing estradiol production in Fshb knockout mice (FIG. 1B).

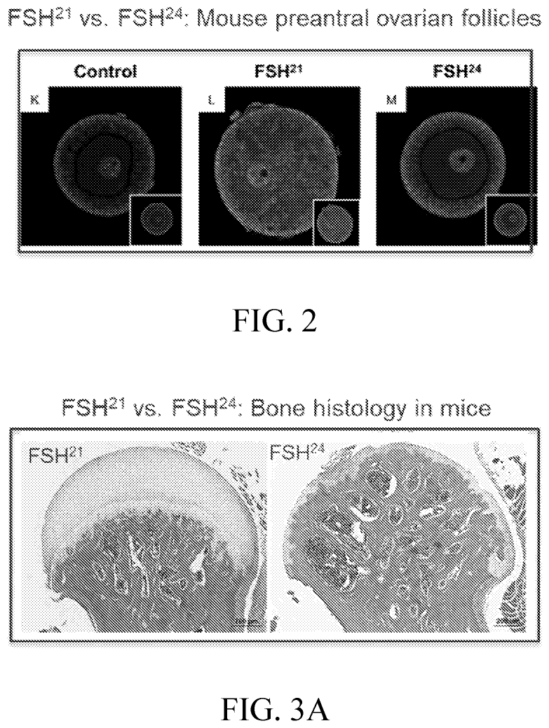

Induction of preantral ovarian follicle growth by this recombinant FSH²¹ was compared to FSH²⁴, and the results of this examination are shown in FIG. 2. These results show that this recombinant FSH²¹ is more effective at inducing gene expression in preantral ovarian follicle production than full-glycosylated FSH²⁴.

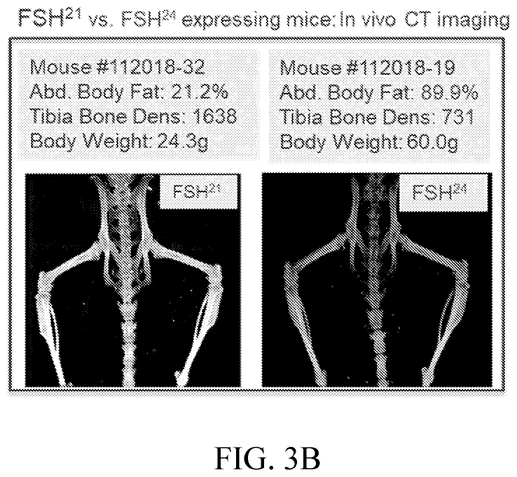

Bone health in mice expressing this recombinant FSH²¹ was compared to mice expressing FSH²⁴, and the results of this examination are shown in FIGS. 3A and 3B. Histological examination by hematoxylin and eosin staining (FIG. 3A) and CT imaging (FIG. 3B) both show mice expressing this recombinant FSH²¹ exhibit greater bone density than mice expressing full-glycosylated FSH²⁴.

### 2. Bone Quality in Mice Expressing Hypo-Glycosylated FSH Compared to Fully Glycosylated FSH

Generation of FSHβ²¹ and FSHβ¹⁸ expressing mice: The human FSHB wild-type gene (encodes FSHβ²⁴) was separately PCR mutagenized at nucleotides coding for Asn²⁴ (codes for FSHβ¹) and Asn⁷ (codes for FSHβ¹⁸). The purified transgene DNA fragments were separately microinjected into one-cell mouse embryos and transgene founders for produced. After stably propagating the transgene into F2 generation, individual lines were introduced onto Fshb null genetic background to derive FSH²⁴, FSH²¹ and FSH¹⁸ expressing mice.

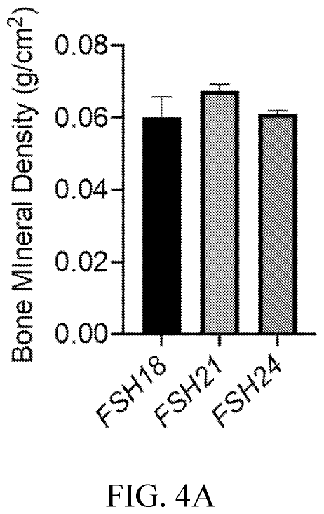

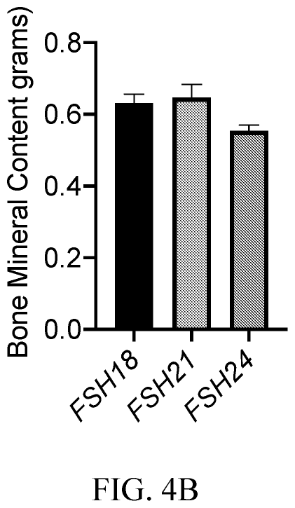

Bone mineral density and content of female mice expressing FSH¹⁸, FHS²¹, or FSH²⁴ glycoforms was examined at 8 months of age (N=11-17) using in vivo dual-energy X-ray absorptiometry (DEXA, or DXA). The results of this examination are shown for bone mineral density in FIG. 4A and bone mineral content in FIG. 4B. These results indicate that mice expressing FSH²¹ and FSH¹⁸, hypo-glycosylated forms of FSH-β, particularly mice expressing FSH²¹, exhibit higher bone mineral density and bone mineral content (P<0.05) than mice expressing fully glycosylated (FSH²⁴) forms of FSH-β.

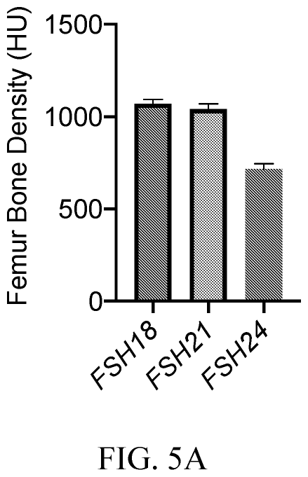

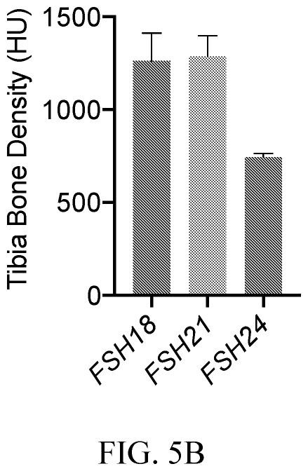

Bone density of the femur and tibia of female mice expressing FSH¹⁸, FHS²¹, or FSH²⁴ glycoforms was examined at 8 months of age (N=3-7) using in vivo X-ray computed tomography (CT) analysis. The results of this examination are shown in FIGS. 5A and 5B. These results indicate that mice expressing FSH²¹ and FSH¹⁸, hypo-glycosylated forms of FSH-β, exhibit higher bone density in both the femur (FIG. 5A) and tibia (FIG. 5B) (P<0.05) than mice expressing fully-glycosylated (FSH²⁴) forms of FSH-β. 3. Fat accumulation in mice expressing hypo-glycosylated FSH compared to fully glycosylated FSH

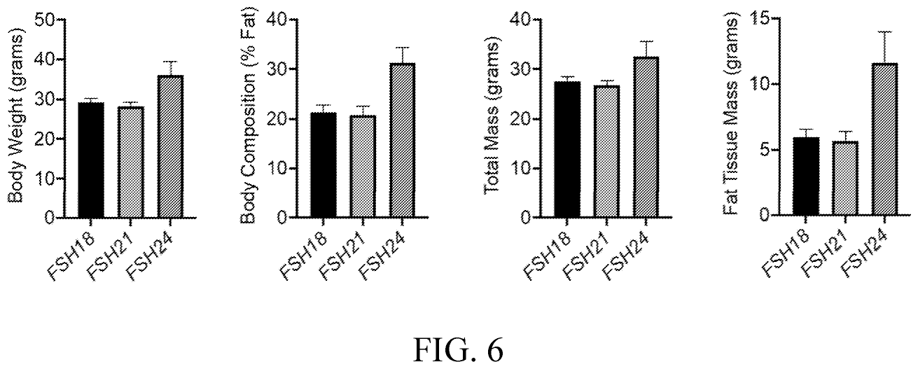

Body weight, fat composition, total mass and fatty tissue mass of female mice expressing FSH¹⁸, FHS²¹, or FSH²⁴ glycoforms was examined at 8 months of age (N=11-17) using in vivo DEXA. The results of this examination are shown in FIG. 6. These results indicate that mice expressing FSH²¹ and FSH¹⁸, hypo-glycosylated forms of FSH-β, exhibit lower body weight, fat composition, indicated by % body weight of fat, total body mass, and total fat mass (P<0.05) than mice expressing fully glycosylated (FSH²⁴) forms of FSH-β.

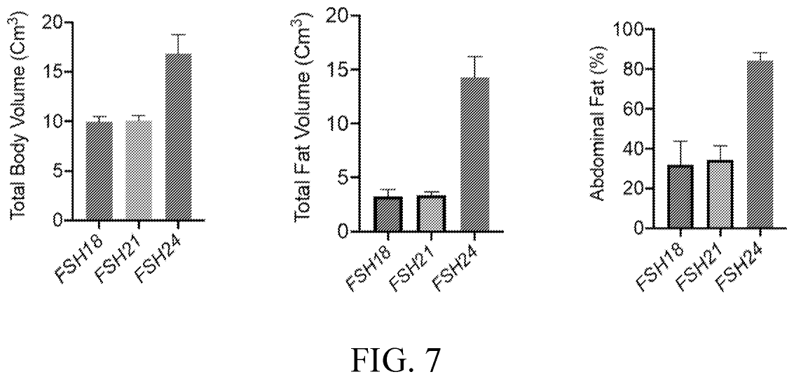

Total body volume, total fat volume, and abdominal fat percentage of female mice expressing FSH¹⁸, FHS²¹, or FSH²⁴ glycoforms was examined at 8 months of age (N=3-7) using in vivo MRI analysis. The results of this examination are shown in FIG. 7. These results indicate that mice expressing FSH²¹ and FSH¹⁸, hypo-glycosylated forms of FSH-β, exhibit lower total body volume, total fat volume, and percentage abdominal fat (P<0.05) than mice expressing fully glycosylated (FSH²⁴) forms of FSH-β.

The foregoing is intended to be illustrative of the present invention and is not to be construed as limiting thereof. The invention is defined by the following claims, with equivalents of the claims to be included therein.

1. A recombinant Follicle-Stimulating Hormone (FSH) beta-subunit, wherein a glycosylation site on the beta-subunit is substituted with an amino acid at which a glycan is not and/or cannot be attached.

2. The recombinant FSH beta-subunit of claim 1, wherein the glycosylation site that is substituted is Asn-7 or Asn-24 of an FSH beta-subunit of human FSH, or its equivalent.

3. The recombinant FSH beta-subunit of claim 2, wherein the glycosylation site that is substituted is Asn-24 of an FSH beta-subunit of human FSH, or its equivalent.

4. The recombinant FSH beta-subunit of claim 1, wherein the glycosylation site that is substituted is substituted with an Alanine or Glutamine.

5. The recombinant FSH beta-subunit of claim 1, wherein the FSH beta-subunit comprises a glycan at Asn-7 of an FSH beta-subunit of human FSH, or its equivalent.

6. The recombinant FSH beta-subunit of claim 1, wherein the beta-subunit comprises an amino acid sequence of SEQ ID NO:3 or SEQ ID NO:4:
(SEQ ID NO: 3)
NSCELTNITI AIEKEECRFC ISIATTWCAG YCYTRDLVYK
DPARPKIQKT CTFKELVYET VRVPGCAHHA DSLYTYPVAT
QCHCGKCDSD STDCTVRGLG PSYCSFGEMK E
(SEQ ID NO: 4)
NSCELTNITI AIEKEECRFC ISIQTTWCAG YCYTRDLVYK
DPARPKIQKT CTFKELVYET VRVPGCAHHA DSLYTYPVAT
QCHCGKCDSD STDCTVRGLG PSYCSFGEMK E.

7. The recombinant FSH beta-subunit of claim 1, wherein the beta-subunit further comprises 1-30 additional amino acids attached to the N-terminal and/or C-terminal of the beta-subunit.

8. The recombinant FSH beta-subunit of claim 7, wherein the 1-30 additional amino acids attached to the N-terminal and/or C-terminal of the beta-subunit comprise sequences from a gonadotropin hormone attached to the N-terminal or C-terminal of the beta-subunit.

9. The recombinant FSH beta-subunit of claim 7, wherein the 1-30 additional amino acids are attached to the C-terminal of the beta-subunit.

10. The recombinant FSH beta-subunit ofany one of m claim 7, wherein the 1-30 additional amino acids comprise a sequence as set forth in SEQ ID NO:11.
(SEQ ID NO: 11)
SSSSKAPPPS LPSPSRLPGP SDTPILPQ.

11. A recombinant Follicle-Stimulating Hormone (FSH) comprising an FSH alpha-subunit and an FSH beta-subunit, wherein a glycosylation site on the beta-subunit is substituted with an amino acid at which a glycan is not and/or cannot be attached.

12. The recombinant FSH of claim 11, wherein the glycosylation site that is substituted is Asn-7 or Asn-24 of an FSH beta-subunit of human FSH, or its equivalent.

13. The recombinant FSH of claim 12, wherein the glycosylation site that is substituted is Asn-24 of an FSH beta-subunit of human FSH, or its equivalent.

14. The recombinant FSH of claim 11, wherein the glycosylation site that is substituted is substituted with an Alanine.

15. The recombinant FSH of claim 11, wherein the recombinant FSH comprises human or modified human FSH.

16. The recombinant FSH of claim 11, wherein the FSH alpha-subunit is fully glycosylated.

17. The recombinant FSH of claim 11, wherein the FSH beta-subunit comprises a glycan at Asn-7 of an FSH beta-subunit of human FSH, or its equivalent.

18. The recombinant FSH of claim 11, wherein the FSH beta-subunit comprises an amino acid sequence of SEQ ID NO:3 or SEQ ID NO:4:
(SEQ ID NO: 3)
NSCELTNITI AIEKEECRFC ISIATTWCAG YCYTRDLVYK
DPARPKIQKT CTFKELVYET VRVPGCAHHA DSLYTYPVAT
QCHCGKCDSD STDCTVRGLG PSYCSFGEMK E
(SEQ ID NO: 4)
NSCELTNITI AIEKEECRFC ISIQTTWCAG YCYTRDLVYK
DPARPKIQKT CTFKELVYET VRVPGCAHHA DSLYTYPVAT
QCHCGKCDSD STDCTVRGLG PSYCSFGEMK E.

19. The recombinant FSH of claim 11, wherein the FSH alpha-subunit and/or FSH beta-subunit further comprises 1-30 additional amino acids attached to the N-terminal and/or C-terminal of the FSH alpha-subunit and/or FSH beta-subunit.

20. The recombinant FSH beta-subunit of claim 19, wherein the 1-30 additional amino acids attached to the N-terminal and/or C-terminal of the beta-subunit comprise sequences from a gonadotropin hormone attached to the N-terminal or C-terminal of the FSH beta-subunit.

21. The recombinant FSH beta-subunit of claim 19, wherein the 1-30 additional amino acids are attached to the C-terminal of the FSH beta-subunit.

22. The recombinant FSH beta-subunit of claim 19, wherein the 1-30 additional amino acid comprise a sequence as set forth in SEQ ID NO:11.
SSSSKAPPPS LPSPSRLPGP SDTPILPQ (SEQ ID NO:11).

23. A nucleic acid comprising a sequence encoding the recombinant FSH beta-subunit of claim 1.

24. The nucleic acid of claim 15, wherein the nucleic acid comprises a nucleotide sequence as set forth in SEQ ID NO:6:
(SEQ ID NO: 6)
AAT AGC TGT GAG CTG ACC AAC ATC ACC ATT GCA ATA
GAG AAA GAA GAA TGT CGT TTC TGC ATA AGC ATC CAG
ACC ACT TGG TGT GCT GGC TAC TGC TAC ACC AGG GAT
CTG GTG TAT AAG GAC CCA GCC AGG CCC AAA ATC CAG
AAA ACA TGT ACC TTC AAG GAA CTG GTA TAC GAA ACA
GTG AGA GTG CCC GGC TGT GCT CAC CAT GCA GAT TCC
TTG TAT ACA TAC CCA GTG GCC ACC CAG TGT CAC TGT
GGC AAG TGT GAC AGC GAC AGC ACT GAT TGT ACT GTG
CGA GGC CTG GGG CCC AGC TAC TGC TCC TTT GGT GAA
ATG AAA GAA.

25. The nucleic acid of claim 16, wherein the nucleic acid comprises a sequence as set forth in SEQ ID NO:14:
(SEQ ID NO: 14)
AAT AGC TGT GAG CTG ACC AAC ATC ACC ATT GCA ATA
GAG AAA GAA GAA TGT CGT TTC TGC ATA AGC ATC CAG
ACC ACT TGG TGT GCT GGC TAC TGC TAC ACC AGG GAT
CTG GTG TAT AAG GAC CCA GCC AGG CCC AAA ATC CAG
AAA ACA TGT ACC TTC AAG GAA CTG GTA TAC GAA ACA
GTG AGA GTG CCC GGC TGT GCT CAC CAT GCA GAT TCC
TTG TAT ACA TAC CCA GTG GCC ACC CAG TGT CAC TGT
GGC AAG TGT GAC AGC GAC AGC ACT GAT TGT ACT GTG
CGA GGC CTG GGG CCC AGC TAC TGC TCC TTT GGT GAA
ATG AAA GAA TCC TCT TCC TCA AAG GCC CCT CCC CCC
AGC CTT CCA AGC CCA TCC CGA CTC CCG GGG CCC TCG
GAC ACC CCG ATC CTC CCA CAA.

26. The nucleic acid of claim 15, wherein the nucleic acid comprises a nucleotide sequence as set forth in SEQ ID NO:10:
(SEQ ID NO: 10)
AAT AGC TGT GAG CTG ACC CAG ATC ACC ATT GCA ATA
GAG AAA GAA GAA TGT CGT TTC TGC ATA AGC ATC AAC
ACC ACT TGG TGT GCT GGC TAC TGC TAC ACC AGG GAT
CTG GTG TAT AAG GAC CCA GCC AGG CCC AAA ATC CAG
AAA ACA TGT ACC TTC AAG GAA CTG GTA TAC GAA ACA
GTG AGA GTG CCC GGC TGT GCT CAC CAT GCA GAT TCC
TTG TAT ACA TAC CCA GTG GCC ACC CAG TGT CAC TGT
GGC AAG TGT GAC AGC GAC AGC ACT GAT TGT ACT GTG
CGA GGC CTG GGG CCC AGC TAC TGC TCC TTT GGT GAA
ATG AAA GAA.

27. The nucleic acid of claim 18, wherein the nucleic acid comprises a sequence as set forth in SEQ ID NO:17:
(SEQ ID NO: 17)
AAT AGC TGT GAG CTG ACC CAG ATC ACC ATT GCA ATA
GAG AAA GAA GAA TGT CGT TTC TGC ATA AGC ATC AAC
ACC ACT TGG TGT GCT GGC TAC TGC TAC ACC AGG GAT
CTG GTG TAT AAG GAC CCA GCC AGG CCC AAA ATC CAG
AAA ACA TGT ACC TTC AAG GAA CTG GTA TAC GAA ACA
GTG AGA GTG CCC GGC TGT GCT CAC CAT GCA GAT TCC
TTG TAT ACA TAC CCA GTG GCC ACC CAG TGT CAC TGT
GGC AAG TGT GAC AGC GAC AGC ACT GAT TGT ACT GTG
CGA GGC CTG GGG CCC AGC TAC TGC TCC TTT GGT GAA
ATG AAA GAA TCC TCT TCC TCA AAG GCC CCT CCC CCC
AGC CTT CCA AGC CCA TCC CGA CTC CCG GGG CCC TCG
GAC ACC CCG ATC CTC CCA CAA.

28. The nucleic acid of any one of claim 23, further comprising a heterologous promoter operably associated therewith.

29. An expression vector comprising the nucleic acid of claim 23.

30. A cell line comprising the nucleic acid or expression vector of claim 23.

31. A cell line expressing the recombinant FSH beta-subunit of claim 1.

32. The cell line of claim 30, wherein the cell line is the cell line is GH3 derived from a rat pituitary gland tumor.

33. A pharmaceutical composition comprising the FSH beta-subunit of claim 1, and a pharmaceutically acceptable carrier.

34. The pharmaceutical composition of claim 33, wherein the composition is suitable for subcutaneous or intravenous administration.

35. A method of treating infertility comprising administering a therapeutically effective amount of the pharmaceutical composition of claim 33 to a subject in need thereof.

36. The method of claim 31, wherein treatment is selected from any one of the group consisting of assisted reproductive technologies (ART), induction of follicle growth and/or maturation, induction of ovular growth and/or maturation, induction of ovulation, and in vitro fertilization, or any combination thereof.

37. A method of inducing follicle growth and/or maturation comprising administration of the pharmaceutical composition of claim 33 to a subject in need thereof.

38. A method of inducing oocyte/egg growth and/or maturation comprising administration of the pharmaceutical composition of claim 33 to a subject in need thereof.

39. A method of stimulating secretion of a sex steroid comprising administering a therapeutically effective amount of the pharmaceutical composition of claim 33 to a subject in need thereof.

40. The method of claim 39, wherein the sex steroid is an estrogen.

41. The method of claim 39, wherein the sex steroid is estradiol or progesterone.

42. A method of treating and/or preventing loss of bone density comprising administering a therapeutically effective amount of the pharmaceutical composition of claim 33 to a subject in need thereof.

43. The method of claim 42, wherein treatment comprises treating osteoporosis.

44. A method of treating and/or preventing fatty tissue accumulation comprising administering a therapeutically effective amount of the pharmaceutical composition of claim 33 to a subject in need thereof.

45. A method of inducing or improving folliculogenesis comprising administering a therapeutically effective amount of the pharmaceutical composition of claim 33 to a subject in need thereof.

46. The method of claim 35, wherein the pharmaceutical composition is administered subcutaneously or intravenously.

47. The method of claim 35, wherein the subject is a human subject.

48. The method of claim 47, wherein the human subject is a female human subject.

49. The method of claim 48, wherein the female human subject is a perimenopausal or menopausal subject.

50. A method of inducing or improving spermatogenesis comprising administering a therapeutically effective amount of the pharmaceutical composition of claim 33 to a subject in need thereof.

51. A method of inducing or improving Sertoli cell proliferation comprising administering a therapeutically effective amount of the pharmaceutical composition of claim 33 to a subject in need thereof.

52. A method of treating hypogonadotropic hypogonadism comprising administering a therapeutically effective amount of the pharmaceutical composition of claim 33 to a subject in need thereof.

53. The method of claim 50, wherein the subject is a human subject.

54. The method of claim 53, wherein the human subject is a male human subject.


SEQUENCE LISTING (17 sequences)

SEQ ID NO: 1 - protein, 92 aa, Homo sapiens
APDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 2 - protein, 111 aa, Homo sapiens
NSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 3 - protein, 111 aa, synthetic construct
NSCELTNITIAIEKEECRFCISIATTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 4 - protein, 111 aa, synthetic construct
NSCELTNITIAIEKEECRFCISIQTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 5 - DNA, 387 bp, synthetic construct
  source 1..387: mol_type = other DNA
atgaagacactccagtttttcttccttttctgttgctggaaagcaatctgctgcaatagctgtgagctgaccaacatcaccattgcaatagagaaagaagaatgtcgtttctgcataagcatccagaccacttggtgtgctggctactgctacaccagggatctggtgtataaggacccagccaggcccaaaatccagaaaacatgtaccttcaaggaactggtatacgaaacagtgagagtgcccggctgtgctcaccatgcagattccttgtatacatacccagtggccacccagtgtcactgtggcaagtgtgacagcgacagcactgattgtactgtgcgaggcctggggcccagctactgctcctttggtgaaatgaaagaa

SEQ ID NO: 6 - DNA, 333 bp, synthetic construct
  source 1..333: mol_type = other DNA
aatagctgtgagctgaccaacatcaccattgcaatagagaaagaagaatgtcgtttctgcataagcatccagaccacttggtgtgctggctactgctacaccagggatctggtgtataaggacccagccaggcccaaaatccagaaaacatgtaccttcaaggaactggtatacgaaacagtgagagtgcccggctgtgctcaccatgcagattccttgtatacatacccagtggccacccagtgtcactgtggcaagtgtgacagcgacagcactgattgtactgtgcgaggcctggggcccagctactgctcctttggtgaaatgaaagaa

SEQ ID NO: 7 - protein, 111 aa, synthetic construct
NSCELTAITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 8 - protein, 111 aa, synthetic construct
NSCELTQITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 9 - DNA, 387 bp, synthetic construct
  source 1..387: mol_type = other DNA
atgaagacactccagtttttcttccttttctgttgctggaaagcaatctgctgcaatagctgtgagctgacccagatcaccattgcaatagagaaagaagaatgtcgtttctgcataagcatcaacaccacttggtgtgctggctactgctacaccagggatctggtgtataaggacccagccaggcccaaaatccagaaaacatgtaccttcaaggaactggtatacgaaacagtgagagtgcccggctgtgctcaccatgcagattccttgtatacatacccagtggccacccagtgtcactgtggcaagtgtgacagcgacagcactgattgtactgtgcgaggcctggggcccagctactgctcctttggtgaaatgaaagaa

SEQ ID NO: 10 - DNA, 333 bp, synthetic construct
  source 1..333: mol_type = other DNA
aatagctgtgagctgacccagatcaccattgcaatagagaaagaagaatgtcgtttctgcataagcatcaacaccacttggtgtgctggctactgctacaccagggatctggtgtataaggacccagccaggcccaaaatccagaaaacatgtaccttcaaggaactggtatacgaaacagtgagagtgcccggctgtgctcaccatgcagattccttgtatacatacccagtggccacccagtgtcactgtggcaagtgtgacagcgacagcactgattgtactgtgcgaggcctggggcccagctactgctcctttggtgaaatgaaagaa

SEQ ID NO: 11 - protein, 28 aa, Homo sapiens
SSSSKAPPPSLPSPSRLPGPSDTPILPQ

SEQ ID NO: 12 - protein, 139 aa, synthetic construct
NSCELTNITIAIEKEECRFCISIQTTWCAGYCYTRDLVYKDPARPKIQKTCTFKELVYETVRVPGCAHHADSLYTYPVATQCHCGKCDSDSTDCTVRGLGPSYCSFGEMKESSSSKAPPPSLPSPSRLPGPSDTPILPQ

SEQ ID NO: 13 - DNA, 471 bp, synthetic construct
  source 1..471: mol_type = other DNA
atgaagacactccagtttttcttccttttctgttgctggaaagcaatctgctgcaatagctgtgagctgaccaacatcaccattgcaatagagaaagaagaatgtcgtttctgcataagcatccagaccacttggtgtgctggctactgctacaccagggatctggtgtataaggacccagccaggcccaaaatccagaaaacatgtaccttcaaggaactggtatacgaaacagtgagagtgcccggctgtgctcaccatgcagattccttgtatacatacccagtggccacccagtgtcactgtggcaagtgtgacagcgacagcactgattgtactgtgcgaggcctggggcccagctactgctcctttggtgaaatgaaagaatcctcttcctcaaaggcccctccccccagccttccaagcccatcccgactcccggggccctcggacaccccgatcctcccacaa

SEQ ID NO: 14 - DNA, 417 bp, synthetic construct
  source 1..417: mol_type = other DNA
aatagctgtgagctgaccaacatcaccattgcaatagagaaagaagaatgtcgtttctgcataagcatccagaccacttggtgtgctggctactgctacaccagggatctggtgtataaggacccagccaggcccaaaatccagaaaacatgtaccttcaaggaactggtatacgaaacagtgagagtgcccggctgtgctcaccatgcagattccttgtatacatacccagtggccacccagtgtcactgtggcaagtgtgacagcgacagcactgattgtactgtgcgaggcctggggcccagctactgctcctttggtgaaatgaaagaatcctcttcctcaaaggcccctccccccagccttccaagcccatcccgactcccggggccctcggacaccccgatcctcccacaa

SEQ ID NO: 15 - protein, 139 aa, synthetic construct
NSCELTQITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKESSSSKAPPPSLPSPSRLPGPSDTPILPQ

SEQ ID NO: 16 - DNA, 471 bp, synthetic construct
  source 1..471: mol_type = other DNA
atgaagacactccagtttttcttccttttctgttgctggaaagcaatctgctgcaatagctgtgagctgacccagatcaccattgcaatagagaaagaagaatgtcgtttctgcataagcatcaacaccacttggtgtgctggctactgctacaccagggatctggtgtataaggacccagccaggcccaaaatccagaaaacatgtaccttcaaggaactggtatacgaaacagtgagagtgcccggctgtgctcaccatgcagattccttgtatacatacccagtggccacccagtgtcactgtggcaagtgtgacagcgacagcactgattgtactgtgcgaggcctggggcccagctactgctcctttggtgaaatgaaagaatcctcttcctcaaaggcccctccccccagccttccaagcccatcccgactcccggggccctcggacaccccgatcctcccacaa

SEQ ID NO: 17 - DNA, 417 bp, synthetic construct
  source 1..417: mol_type = other DNA
aatagctgtgagctgacccagatcaccattgcaatagagaaagaagaatgtcgtttctgcataagcatcaacaccacttggtgtgctggctactgctacaccagggatctggtgtataaggacccagccaggcccaaaatccagaaaacatgtaccttcaaggaactggtatacgaaacagtgagagtgcccggctgtgctcaccatgcagattccttgtatacatacccagtggccacccagtgtcactgtggcaagtgtgacagcgacagcactgattgtactgtgcgaggcctggggcccagctactgctcctttggtgaaatgaaagaatcctcttcctcaaaggcccctccccccagccttccaagcccatcccgactcccggggccctcggacaccccgatcctcccacaa